SEQUENCE LISTING (3 sequences)

SEQ ID NO: 1 - DNA, 23 bp, Artificial
  Synthetic
tgcaggaaggggaggaggggctg

SEQ ID NO: 2 - DNA, 23 bp, Artificial
  Synthetic
tgcaaggaggggaggagggcccg

SEQ ID NO: 3 - DNA, 26 bp, Artificial
  Synthetic
ccccagtttagtagttggacttaggg

(19) United States
(12) Patent Application Publication
(10) Pub. No.: US 2005/0003369 A1
(43) Pub. Date: Jan. 6, 2005

(54) Method for depleting specific nucleic acids from a mixture

(75) Inventors: Frederick C. Christians, Los Altos, CA (US); Rui Mei, Santa Clara, CA (US); Kai Wu, Mountain View, CA (US); Charles G. Miyada, San Jose, CA (US)

Correspondence Address: AFFYMETRIX, INC;ATTN: CHIEF IP COUNSEL, LEGAL DEPT., 3380 CENTRAL EXPRESSWAY, SANTA CLARA, CA, 95051, US

(73) Assignee: Affymetrix, INC., Santa Clara, CA (US)

(21) Appl. No.: 10/684,205

(22) Filed: Oct. 10, 2003

Related U.S. Application Data
(60) Provisional application No. 60/417,803, filed on Oct. 10, 2002.
(60) Provisional application No. 60/417,817, filed on Oct. 11, 2002.

(51) Int. Cl. C12Q 1/68

(52) U.S. Cl. 435/6

(57) ABSTRACT

The presently claimed invention provides methods, compositions, and apparatus for analyzing nucleic acids isolated from blood. Specifically, the present invention provides a method of analyzing blood samples by blocking amplification of selected unwanted RNAs and subsequently analyzing the amplified sample by hybridization to a plurality of probes attached to a solid support. In one embodiment, the invention provides enriching for a population of interest in a complex population by diminishing the presence of an unwanted sequence that may interfere with the analysis of sequences of interest.

### RELATED APPLICATIONS

This application claims priority to provisional applications 60/417,803 filed Oct. 10, 2002 and 60/417,817 filed Oct. 11, 2002, the disclosures of which are incorporated herein by reference in their entireties.

### FIELD OF THE INVENTION

The present invention relates generally to the amplification of nucleic acids. More specifically, the present invention facilitates the amplification of target mRNA while reducing amplification of unwanted mRNA. The amplified mRNA may be used for a variety of end uses.

### BRIEF DESCRIPTION OF THE FIGURE

FIG. 1A shows a schematic of one embodiment. A population of mRNA comprising a mixture of globin mRNA and target mRNA is mixed with oligonucleotides that are complementary to the globin mRNAs just upstream of the polyA tail (globin reduction oligonucleotides, GROs). The reduction oligonucleotides form RNA:DNA hybrids with globin mRNAs but not with mRNAs that are to be amplified (target mRNAs). RNase H is added to the mixture and the RNA in the RNA:DNA hybrid is cleaved. RNase H is removed or inactivated.

FIG. 1B the target mRNA is reverse transcribed using an oligo(dT)-T7 promoter primer and the first strand cDNA is converted to ds-cDNA with a T7 promoter. The ds-cDNA is used to transcribe cRNA of the target mRNA. The cRNA is the complement of the target mRNA.

### SUMMARY OF THE INVENTION

The presently claimed invention provides methods of preparing a nucleic acid sample for analysis by depletion of selected mRNAs. The remaining RNA in the sample may then be analyzed by a variety of methods, including by hybridization to an array of nucleic acid probes.

In one embodiment, the presently claimed invention provides a method of preparing a nucleic acid sample for analysis comprising enriching for a population of interest within a mixed population of nucleic acids by contacting the nucleic acid sample with a bait molecule (reduction oligonucleotide) that hybridizes to sequences that are not targeted for amplification. The bait molecule is capable of complexing specifically to unwanted sequences within the nucleic acid sample, but is incapable of complexing with sequences of interest. The bait molecule is contacted with the unwanted sequences forming RNA:DNA complexes. Formation of the complex of the bait molecule and the unwanted sequence is used to remove the unwanted sequence from the population of RNAs that will be efficiently amplified in subsequent steps. The remaining enriched population of interest is then amplified resulting in enrichment of the sequences of interest relative to the unwanted sequences. The samples are preferably blood samples or other samples that have a high level of specific unwanted RNAs.

In one embodiment the complex of the bait molecule and the unwanted sequence is incubated with RNase H, resulting in cleavage of the unwanted sequence. The cleavage may be targeted to be just upstream of the poly(A) tail of an mRNA so that if the amplification is via priming with oligo(dT) the cleaved unwanted mRNA will not be amplified. The cleavage may be targeted to multiple regions of the unwanted RNA. In one embodiment the complex of the bait molecule and the unwanted sequence is separated from the nucleic acid sample. For example, by affinity chromatography. The unwanted RNAs are thus removed prior to a subsequent amplification step and are not amplified.

In another embodiment, the presently claimed invention provides a method for analyzing eukaryotic mRNA comprising: obtaining a population of RNA from a eukaryotic organism; enriching the population for mRNA by exposing the population to at least one DNA bait molecule which is complementary to a region near the 3′ end of at least one unwanted sequence in said population under such conditions as to allow for the formation of DNA:RNA hybrids; exposing the DNA:RNA hybrids to RNAse H to remove the RNA from said DNA:RNA hybrids and cleave the RNA into a 5′ fragment and a 3′ fragment; hybridizing a primer comprising oligo dT to the RNA population and extending the primer, thus producing an amplified population of mRNA

### DETAILED DESCRIPTION

A. General

The present invention has many preferred embodiments and relies on many patents, applications and other references for details known to those of the art. Therefore, when a patent, application, or other reference is cited or repeated below, it should be understood that it is incorporated by reference in its entirety for all purposes as well as for the proposition that is recited.

As used in this application, the singular form “a,” “an,” and “the” include plural references unless the context clearly dictates otherwise. For example, the term “an agent” includes a plurality of agents, including mixtures thereof.

An individual is not limited to a human being but may also be other organisms including but not limited to mammals, plants, bacteria, or cells derived from any of the above.

Throughout this disclosure, various aspects of this invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

The practice of the present invention may employ, unless otherwise indicated, conventional techniques and descriptions of organic chemistry, polymer technology, molecular biology (including recombinant techniques), cell biology, biochemistry, and immunology, which are within the skill of the art. Such conventional techniques include polymer array synthesis, hybridization, ligation, and detection of hybridization using a label. Specific illustrations of suitable techniques can be had by reference to the example herein below. However, other equivalent conventional procedures can, of course, also be used. Such conventional techniques and descriptions can be found in standard laboratory manuals such as Genome Analysis: A Laboratory Manual Series (Vols. I-IV), Using Antibodies: A Laboratory Manual, Cells: A Laboratory Manual, PCR Primer: A Laboratory Manual, and Molecular Cloning: A Laboratory Manual (all from Cold Spring Harbor Laboratory Press), Stryer, L. (1995) Biochemistry (4th Ed.) Freeman, New York, Gait, “Oligonucleotide Synthesis: A Practical Approach” 1984, IRL Press, London, Nelson and Cox (2000), Lehninger, Principles of Biochemistry 3^{rd} Ed., W. H. Freeman Pub., New York, N.Y. and Berg et al. (2002) Biochemistry, 5^{th} Ed., W. H. Freeman Pub., New York, N.Y. all of which are herein incorporated in their entirety by reference for all purposes.

The present invention can employ solid substrates, including arrays in some preferred embodiments. Methods and techniques applicable to polymer (including protein) array synthesis have been described in U.S.S.N 09/536,841, WO 00/58516, U.S. Pat. Nos. 5,143,854, 5,242,974, 5,252,743, 5,324,633, 5,384,261, 5,424,186, 5,451,683, 5,482,867, 5,491,074, 5,527,681, 5,550,215, 5,571,639, 5,578,832, 5,593,839, 5,599,695, 5,624,711, 5,631,734, 5,795,716, 5,831,070, 5,837,832, 5,856,101, 5,858,659, 5,936,324, 5,968,740, 5,974,164, 5,981,185, 5,981,956, 6,025,601, 6,033,860, 6,040,193, 6,090,555, and 6,136,269, in PCT Applications Nos. PCT/US99/00730 (International Publication Number WO 99/36760) and PCT/US 01/04285, and in U.S. patent applications Ser. Nos. 09/501,099 and 09/122,216 which are all incorporated herein by reference in their entirety for all purposes.

Patents that describe synthesis techniques in specific embodiments include U.S. Pat. Nos. 5,412,087, 6,147,205, 6,262,216, 6,310,189, 5,889,165, and 5,959,098. Nucleic acid arrays are described in many of the above patents, but the same techniques are applied to polypeptide arrays.

The present invention also contemplates many uses for polymers attached to solid substrates. These uses include gene expression monitoring, profiling, library screening, genotyping, and diagnostics. Gene expression monitoring and profiling methods can be shown in U.S. Pat. Nos. 5,800,992, 6,013,449, 6,020,135, 6,033,860, 6,040,138, 6,177,248 and 6,309,822. Genotyping and uses therefore are shown in U.S. Ser. No. 10/013,598, and U.S. Pat. Nos. 5,856,092, 6,300,063, 5,858,659, 6,284,460, 6,361,947, 6,368,799 and 6,333,179. Other uses are embodied in U.S. Pat. Nos. 5,871,928, 5,902,723, 6,045,996, 5,541,061, and 6,197,506.

The present invention also contemplates sample preparation methods in certain preferred embodiments. For example, see the patents in the gene expression, profiling, genotyping and other use patents above, as well as U.S. Ser. No. 09/854,317, Wu and Wallace, Genomics 4, 560 (1989), Landegren et al., Science 241, 1077 (1988), Burg, U.S. Pat. Nos. 5,437,990, 5,215,899, 5,466,586, 4,357,421, Gubler et al., 1985, Biochemica et Biophysica Acta, Displacement Synthesis of Globin Complementary DNA: Evidence for Sequence Amplification, transcription amplification, Kwoh et al., Proc. Natl. Acad. Sci. USA 86, 1173 (1989), Guatelli et al., Proc. Nat. Acad. Sci. USA, 87, 1874 (1990), WO 88/10315, WO 90/06995, and 6,361,947.

The present invention also contemplates detection of hybridization between ligands in certain preferred embodiments. See U.S. Pat. Nos. 5,143,854, 5,578,832; 5,631,734; 5,834,758; 5,936,324; 5,981,956; 6,025,601; 6,141,096; 6,185,030; 6,201,639; 6,218,803; and 6,225,625 and in PCT Application PCT/US99/06097 (published as WO99/47964), each of which also is hereby incorporated by reference in its entirety for all purposes.

The present invention may also make use of various computer program products and software for a variety of purposes, such as probe design, management of data, analysis, and instrument operation. See, U.S. Pat. Nos. 5,593,839, 5,795,716, 5,733,729, 5,974,164, 6,066,454, 6,090,555, 6,185,561, 6,188,783, 6,223,127, 6,229,911 and 6,308,170. Additionally, the present invention may have preferred embodiments that include methods for providing genetic information over the internet. See provisional application 60/349,546.

B. Definitions

The phrase “massively parallel screening” refers to the simultaneous screening of at least about 100, preferably about 1000, more preferably about 10,000 and most preferably about 1,000,000 different nucleic acid hybridizations.

Nucleic acids according to the present invention may include any polymer or oligomer of pyrimidine and purine bases, preferably cytosine, thymine, and uracil, and adenine and guanine, respectively. (See Albert L. Lehninger, Principles of Biochemistry, at 793-800 (Worth Pub. 1982) which is herein incorporated in its entirety for all purposes). Indeed, the present invention contemplates any deoxyribonucleotide, ribonucleotide or peptide nucleic acid component, and any chemical variants thereof, such as methylated, hydroxymethylated or glucosylated forms of these bases, and the like. The polymers or oligomers may be heterogeneous or homogeneous in composition, and may be isolated from naturally occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states.

An “oligonucleotide” or “polynucleotide” is a nucleic acid ranging from at least 2, preferably at least 8, 15, or 20 to 25 nucleotides in length, but may be up to 50, 100, 1000, or 5000 nucleotides long or a compound that specifically hybridizes to a polynucleotide. Polynucleotides of the present invention include sequences of deoxyribonucleic acid (DNA) or ribonucleic acid (RNA) or mimetics thereof which may be isolated from natural sources, recombinantly produced or artificially synthesized. A further example of a polynucleotide of the present invention may be a peptide nucleic acid (PNA). (See U.S. Pat. No. 6,156,501 which is hereby incorporated by reference in its entirety.) The invention also encompasses situations in which there is a nontraditional base pairing such as Hoogsteen base pairing which has been identified in certain tRNA molecules and postulated to exist in a triple helix. “Polynucleotide” and “oligonucleotide” are used interchangeably in this application.

Oligonucleotides may be chemically synthesized and may include modifications. Amino modifier reagents may be used to introduce a primary amino group into the oligo. A primary amino group is useful for a variety of coupling reactions that can be used to attach various labels to the oligo. The most frequently used labels are in the form of NHS-esters, which can couple with primary amino groups. A variety of derivatives of biotin are available in which the biotin moiety is connected (through the 4-carboxybutyl group) to a linker molecule that can be attached directly to an oligonucleotide. Fluorescent dies such as 6-FAM, HEX, TET, TAMRA, and ROX may be coupled to an oligo. Phosphate groups may be attached to the 5′ and/or 3′ end of an oligo. Oligos may also be phosphorothioated. A phosphorothioate group is a modified phosphate group with one of the oxygen atoms replaced by a sulfur atom. In a phosphorothioated oligo (often called an “S-Oligo”), some or all of the internucleotide phosphate groups are replaced by phosphorothioate groups. The modified “backbone” of an S-Oligo is resistant to the action of most exonucleases and endonucleases. In some embodiments the oligo is sulfurized only at the last few residues at each end of the oligo. This results in an oligo that is resistent to exonucleases, but has a natural DNA center. Degenerate bases may also be incorporated into an oligo. may also be incorporated into an oligo Additional modifications that are available include, for example, 2′O-Methyl RNA, 3′-Glyceryl, 3′-Terminators, Acrydite, Cholesterol labeling, Dabcyl, Digoxigenin labeling, Methylated nucleosides, Spacer Reagents, Thiol Modifications Deoxylnosine, DeoxyUridine and halogenated nucleosides.

A reduction oligonucleotide is an oligonucleotide that is complementary to an unwanted nucleic acid. For example, SEQ ID NOs 1, 2 and 3 may be used as reduction oligos targeting unwanted globin mRNAs.

“Subsequence” refers to a sequence of nucleic acids that comprise a part of a longer sequence of nucleic acids.

The phrase “hybridizing specifically to” refers to the binding, duplexing, or hybridizing of a molecule substantially to or only to a particular nucleotide sequence or sequences under stringent conditions when that sequence is present in a complex mixture (e.g., total cellular) DNA or RNA. Standard conditions are described in, for example, Sambrook, Fritsch, Maniatis “Molecular Cloning: A Laboratory Manual” (1989) Cold Spring Harbor Press.

The term “mRNA” or “mRNA transcripts,” as used herein, include, but not limited to pre-mRNA transcript(s), transcript processing intermediates, mature mRNA(s) ready for translation and transcripts of the gene or genes, or nucleic acids derived from the mRNA transcript(s). Transcript processing may include splicing, editing and degradation. As used herein, a nucleic acid derived from an mRNA transcript refers to a nucleic acid for whose synthesis the mRNA transcript or a subsequence thereof has ultimately served as a template. Thus, a cDNA reverse transcribed from an mRNA, an RNA transcribed from that cDNA, a DNA amplified from the cDNA, an RNA transcribed from the amplified DNA, etc., are all derived from the mRNA transcript and detection of such derived products is indicative of the presence and/or abundance of the original transcript in a sample. Thus, mRNA derived samples include, but are not limited to, mRNA transcripts of the gene or genes, cDNA reverse transcribed from the mRNA, cRNA transcribed from the cDNA, DNA amplified from the genes, RNA transcribed from amplified DNA, and the like.

The term “signal moiety” refers in a general sense to a detectable moiety, such as a radioactive isotope or group containing the same, and non-isotopic moieties, such as enzymes, biotin, avidin, streptavidin, digoxygenin, luminescent agents, dyes, haptens and the like. Luminescent agents, depending upon the source exciting the energy, can be classified as radioluminescent, chemiluminescent, bioluminescent, and photoluminescent (fluorescent).

The phrase “mixed population” or “complex population” refers to any sample containing both desired and undesired nucleic acids. As a non-limiting example, a complex population of nucleic acids may be total genomic DNA, total cellular RNA or a combination thereof. Moreover, a complex population of nucleic acids may have been enriched for a given population but include other undesirable populations. For example, a complex population of nucleic acids may be a sample which has been enriched for desired messenger RNA (mRNA) sequences but still includes some undesired sequences such as ribosomal RNAs (rRNA) or RNAs that are present at disproportionately high levels that may interfere with analysis of other, less abundant, mRNAs.

An “array” comprises a support, preferably solid, with nucleic acid probes attached to the support. Preferred arrays typically comprise a plurality of different nucleic acid probes that are coupled to a surface of a substrate in different, known locations. These arrays, also described as “microarrays” or colloquially “chips” have been generally described in the art, for example, U.S. Pat. Nos. 5,143,854, 5,445,934, 5,744,305, 5,677,195, 5,800,992, 6,040,193, 5,424,186 and Fodor et al., Science, 251:767-777 (1991), each of which is incorporated by reference in its entirety for all purposes.

Arrays may generally be produced using a variety of techniques, such as mechanical synthesis methods or light directed synthesis methods that incorporate a combination of photolithographic methods and solid phase synthesis methods. Techniques for the synthesis of these arrays using mechanical synthesis methods are described in, e.g., U.S. Pat. Nos. 5,384,261, and 6,040,193, which are incorporated herein by reference in their entirety for all purposes. Although a planar array surface is preferred, the array may be fabricated on a surface of virtually any shape or even a multiplicity of surfaces. Arrays may be nucleic acids on beads, gels, polymeric surfaces, fibers such as fiber optics, glass or any other appropriate substrate. (See U.S. Pat. Nos. 5,770,358, 5,789,162, 5,708,153, 6,040,193 and 5,800,992, which are hereby incorporated by reference in their entirety for all purposes.)

Arrays may be packaged in such a manner as to allow for diagnostic use or can be an all-inclusive device; e.g., U.S. Pat. Nos. 5,856,174 and 5,922,591 incorporated in their entirety by reference for all purposes. Preferred arrays are commercially available from Affymetrix (Santa Clara, Calif.) under the brand name GeneChip® and are directed to a variety of purposes, including genotyping and gene expression monitoring for a variety of eukaryotic and prokaryotic species.

Hybridization probes are oligonucleotides capable of binding in a base-specific manner to a complementary strand of nucleic acid. Such probes include peptide nucleic acids, as described in Nielsen et al., Science 254, 1497-1500 (1991), and other nucleic acid analogs and nucleic acid mimetics. See U.S. patent application No. 6,156,501.

Hybridizations are usually performed under stringent conditions, for example, at a salt concentration of no more than 1 M and a temperature of at least 25° C. For example, conditions of 5×SSPE (750 mM NaCl, 50 mM NaPhosphate, 5 mM EDTA, pH 7.4) and a temperature of 25-30° C. are suitable for allele-specific probe hybridizations. For stringent conditions, see, for example, Sambrook et al. which is hereby incorporated by reference in its entirety for all purposes above.

C. Selective Removal of Unwanted Sequences from a Mixture.

Expression profiling with microarrays is an increasingly important tool for clinical research and diagnostics. Blood is a widely used and easily obtained sample type for isolation of RNA. However, blood is composed of heterogeneous cell types. In addition there are a plurality of methods for handling and processing blood and variability may result in changes in the expression profile that are a reflection more of handling method than of patient expression profile. Methods of handling and processing blood for gene expression analysis while preserving expression patterns are disclosed.

In a preferred embodiment RNA is isolated from whole blood using the PAXgene™ Blood RNA Isolation System developed by PreAnalytiX, a QIAGEN BD Company. The system provides collection tubes which contain an RNA stabilizing agent that provides improved RNA stability over time with minimum manual manipulation relative to standard blood collection tubes. See, U.S. Pat. Nos. 6,617,170 and 6,602,718 which are incorporated herein by reference in their entireties.

In one embodiment the blood is collected in a container that contains stabilizing agents. The stabilizing agents may prevent degradation of RNA. Stabilizing agents include cationic compounds, detergents, chaotropic salts, ribonuclease inhibitors, chelating agents, and mixtures thereof; phenol, chloroform, acetone, alcohols and mixtures thereof and mercapto-alcohols, di-thio-threitol (DTT), and mixtures thereof. The agents may be gene induction blocking agents.

In another embodiment the globin reduction protocol is performed on RNA isolated from blood using a method of isolation that results in high levels of globin mRNAs. See Affymetrix Technical Note, “An Analysis of Blood Processing Methods to Prepare Samples for GeneChip® Expression Profiling”, 2003, and Affymetrix, Technical Note, “Globin Reduction Protocol: A Method for Processing Whole Blood RNA Samples for Improved Array Results”, 2003, Affymetrix, Inc., Santa Clara, Calif., both of which are incorporated herein by reference in their entireties. Both technical notes are available on the Affymetrix web site, Affymetrix.com.

Many different techniques are used to separate fractions of blood cells prior to RNA isolation. These methods include isolation of RNA from whole blood, the selective lysis of erythrocytes prior to RNA isolation, purification of peripheral blood mononuclear cells (PBMC), and separation of specific cell populations based on characteristic cell surface antigens. Commonly used blood isolation and separation protocols include the PAXgene™ system from PreAnalytiX, QIAamp® RNA Blood Mini Kits from Qiagen, the Ficoll-Hypaque method and BD Vacutainer™-CPT™ Sodium Citrate Tubes (BD-CPT) from Becton-Dickinson. There are a number of different cell types in whole blood, including red blood cells (RBCs), platelets, white blood cells (WBC's) including granulocytes (neutrophils, basophils, eosinophils) and mononuclear cells (lymphocytes and monocytes). Different separation and fractionation techniques result in isolation of different cell types. PAXgene isolates whole blood thus isolating each of the cell types. QIAamp is an erythrocyte lysis method so each of the cell types except for RBCs are isolated. PBMC methods such as the ficoll method and the BD-CPT method result in isolation of the mononuclear cells (lymphocytes and monocytes). In addition specific cell types may be isolated using a variety of methods including positive and negative selection for the specific subset of cells desired. The disclosed methods may be used to reduce amplification of unwanted mRNAs from samples derived from cells isolated by any of these methods.

PBMCs are the most transcriptionally active cells in blood. As a result the PBMC fraction is often used for research areas such as immunology, infectious and cardiovascular diseases, cancer and biomarker research. The fraction may be isolated by centrifuging whole blood in a liquid density step gradient. It typically contains lymphocytes and monocytes and excludes red blood cells and granulotcytes.

Mature RBCs don't contain RNA but reticulocytes, which are immature RBCs, do contain RNA (rRNA, tRNA and mRNA). The most predominant transcript in reticulocytes is globin mRNA. Althought reticulocytes represent only about 0.5-2.0% of the RBCs in a healthy individual, their RNA may contribute up to 70% of total RNA isolated from whole blood. In one embodiment desired cell types may be enriched relative to undesired cell types by fractionation methods. In some embodiments an enrichment step may be followed by a step to reduce amplification of unwanted mRNAs in a sample.

It has been observed that when globin transcripts constitute greater than 20 percent of the total mRNA population of a sample analysis using microarrays, for example, Affymetrix GeneChip arrays, may be adversely impacted. This impact may be reduced by reducing the globin mRNA to less than 20% of the mRNA in the sample. In a preferred embodiment globin mRNA is specifically reduced from up to 70% down to approximately 20% or less in a nucleic acid sample from whole blood. This reduction improves sensitivity and reduces variability in analysis of the sample using microarrays, such as those available from Affymetrix. In a preferred embodiment the blood sample is from a human and the array has probes that are complementary to human genes, for example, the HG-U133 array from Affymetrix. Oligonucleotides are designed to anneal specifically to unwanted transcripts adjacent to the poly-A tails of the transcripts. Following hybridization of the RNA samples with the reduction oligonucleotides, the samples are treated with a nuclease that digests the RNA strand of the RNA:DNA hybrid, such as RNase H, making these transcripts unavailable in the subsequent oligo(dT)-primed reverse transcription reaction. In a preferred embodiment reduction oligos were designed to target the highest expression globin transcripts in human reticulocytes, including alpha1, alpha2 and beta globin. In other embodiments the reduction oligos are designed to hybridize to globin mRNAs from other organisms. The methods may be used to analyze blood from any organism that has blood.

The sequence and concentration of the reduction oligonucleotides, the concentration of RNase H, and the digestion time and temperature may be varied. In many embodiments RNase H is used to cleave RNA in RNA DNA hybrids, however, one of skill in the art will recognize that any method of cleavage that recognizes the complex of the reduction oligo and the unwanted mRNA could be used. For example, the reduction oligo could be modified with a compound that targets cleavage to the regions where it is hybridized.

Gene expression analysis techniques often employ isolation and amplification of ribonucleic acid (RNA) followed by analysis of the amplified sample, which is often labeled. Because of the interest in identifying protein-encoding genes and in examining gene expression levels, it is often desirable to purify or enrich the messenger RNA (mRNA) or some subset of the RNA. The poly-adenine 3′-terminus (poly-A tail) of mRNA from eukaryotic cells can be used as a handle to bind to poly(dT) oligonucleotides, and this method is widely used to identify, purify and or label eukaryotic mRNA. Often complex samples are comprised of high levels of some nucleic acids that are not of interest for subsequent analysis. Removal of these unwanted nucleic acids from the sample by depletion or by modification so that the unwanted nucleic acids are not substrates or templates for amplification may be used to generate an amplified sample for analysis. The method may be applied to a nucleic acid sample prior to any method of amplification known in the art.

A method for depleting RNA species from nucleic acid samples is described. The method may be used, for example, for removing RNA species from cellular RNA samples such as blood. The blood sample may be isolated by any method. In some embodiments the methods are used for forensic analysis of blood. In particular the method may be used to remove globin mRNAs from samples derived from human blood. Applications include, for example, methods to increase the relative abundance of protein-encoding RNAs (mRNAs) by depleting the very abundant RNAs, such as ribosomal RNA, as disclosed in U.S. Pat. No. 6,613,516 which is incorporated herein by reference in its entirety. General methods for depletion are also disclosed in U.S. Pat. Nos. 6,040,138 and 6,391,592 which are both incorporated herein by reference in their entireties.

In another embodiment, methods to remove or modify one or more mRNA species from a RNA sample consisting of numerous different mRNA species, each present in unknown quantities is disclosed. To such an RNA sample, with specific RNAs knocked out or modified so that they will not be amplified efficiently, one or more of the specific RNAs may be added back in known quantities. This procedure provides a method for developing quantitative assays to measure expression of specific genes in a selected background, for example in a native complex background. In some embodiments the RNAs are physically removed. In another embodiment the RNAs are not physically removed but are functionally knocked out or removed, for example, the polyA tails of specific eukaryotic RNAs can be separated from the rest of the RNA molecule, thereby rendering the RNAs unavailable for cDNA synthesis with poly(dT)-primer and a reverse transcriptase.

In one embodiment a complementary single-stranded DNA “bait” molecule or “reduction oligo” is first hybridized to a region of a specific RNA that is complementary to the reduction oligo. In one embodiment the RNA component of the resulting RNA:DNA hybrid may then be hydrolyzed with an enzyme that is specific for RNA:DNA hybrids, for example, RNaseH. The DNA bait can be designed to hybridize to part or all of the RNA to be hydrolyzed. To sever the 3′-polyA tail from the rest of an mRNA, an oligonucleotide directed to a region upstream or 5′ of the polyA tail may be used. In one embodiment the reduction oligo hybridized to the region that is withing 50, 100 or 200 bases of the 5′end of the poly(A) tail. Hybrids of greater length may be used to generate more extensive hydrolysis. Longer DNA bait could comprise, for example: multiple oligonucleotides hybridizing to different regions of the RNA to be hydrolyzed; single-stranded DNA made from phage carrying at least a portion of the sequence; denatured PCR product; denatured plasmid DNA containing at least a portion of the sequence; and complementary DNA made from the RNA molecules to be hydrolyzed using oligonucleotide primers and reverse transcriptase.

In a preferred embodiment a cocktail or mixture of a plurality of different reduction oligos is hybridized to the sample which is then treated with RNaseH. In a preferred embodiment there are at least 3 reduction oligos used simultaneously. In another embodiment there are reduction oligos for 4 to 10, 10 to 20, 20-100 or more different unwanted mRNAs. Depletion of unwanted mRNAs allows for improved amplification and detection of the mRNAs that are of interest.

In one embodiment after hybridization or association of the DNA reduction oligos to the specific RNA to be removed or hydrolyzed followed by RNaseH hydrolysis, the digested RNA may be separated from the rest of the mixture (for example, by size exclusion columns). In another embodiment the hydrolyzed RNA is left in the mixture. In another embodiment the reduction oligo is removed or digested, for example, by DNase I digestion. In another embodiment the bait molecule is left in the mixture. The RNase H may be inactivated, for example, by heat, the addition of EDTA, or by removal using organic extraction or by column purification of the RNA mixture.

The RNA mixture, now physically or functionally depleted of specific unwanted RNA sequences, may be used for a variety of purposes. For example, it may be used as a complex RNA to which specific RNAs are added at known concentrations. Such a mixture may be used, for example, for testing hybridization kinetics of complex samples. The numerous interactions between RNA molecules of the complex sample would be retained, yet some transcripts would be present in known concentrations to serve as controls. These controls may be used to measure hybridization to probes, for example, DNA probes on microarrays, and may be used for comparison of hybridization properties of different sequences. The controls may be added to their “natural” complex RNA environment. In another embodiment subsets of mRNAs may be analyzed, for example mitochondrial RNAs.

Those skilled in the art know there are many ways to synthesize first strand cDNA from mRNA. (See e.g., Sambrook et al., Molecular Cloning: A Laboratory Manual (2nd ed.), Vols. 1-3, Cold Spring Harbor Laboratory, (1989), or Current Protocols in Molecular Biology, F. Ausubel et al., ed. Greene Publishing and Wiley-Interscience, New York (1987)). For example, the first strand cDNA can be synthesized by a reverse transcriptase with a primer. Reverse transcriptases are readily available from many sources and those skilled in the art will know what reverse transcriptase to use for their specific purposes.

Other suitable amplification methods include the ligase chain reaction (LCR) e.g., Wu and Wallace, Genomics 4, 560 (1989) and Landegren et al., Science 241, 1077 (1988), Burg, U.S. Pat. Nos. 5,437,990, 5,215,899, 5,466,586, 4,357,421, Gubler et al., 1985, Biochemica et Biophysica Acta, Displacement Synthesis of Globin Complementary DNA: Evidence for Sequence Amplification, transcription amplification, Kwoh et al., Proc. Natl. Acad. Sci. USA 86, 1173 (1989), self-sustained sequence replication, Guatelli et al., Proc. Nat. Acad. Sci. USA, 87, 1874 (1990) and WO 88/10315 and WO 90/06995 and nucleic acid based sequence amplification (NABSA). The latter two amplification methods include isothermal reactions based on isothermal transcription, which produce both single-stranded RNA (ssRNA) and double-stranded DNA (dsDNA) as the amplification products in a ratio of about 30 or 100 to 1, respectively. Second strand priming can occur by hairpin loop formation, RNAse H digestion products, and the 3′ end of any nucleic acid present in a reaction capable of forming an extensible complex with the first strand DNA.

Methods of amplifying mRNA are further described in U.S. patent application Ser. Nos. 10/090,320, 09/961,709, 09/738,892, 09/746,113, and 09/285,658 each of which is incorporated herein by reference in its entirety for all purposes.

Methods of isolating total mRNA are well known to those of skill in the art. For example, methods of isolation and purification of nucleic acids are described in detail in Chapter 3 of Laboratory Techniques in Biochemistry and Molecular Biology: Hybridization With Nucleic Acid Probes, Part I. Theory and Nucleic Acid Preparation, P. Tijssen, ed. Elsevier, N.Y. (1993) and Chapter 3 of Laboratory Techniques in Biochemistry and Molecular Biology: Hybridization With Nucleic Acid Probes, Part I. Theory and Nucleic Acid Preparation, P. Tijssen, ed. Elsevier, N.Y. (1993)).

In one embodiment, the total RNA is isolated from a given sample using, for example, an acid guanidinium-phenol-chloroform extraction method and polyA⁺ mRNA is isolated by oligo dT column chromatography or by using (dT)n magnetic beads. (See e.g., Sambrook et al., Molecular Cloning: A Laboratory Manual (2nd ed.), Vols. 1-3, Cold Spring Harbor Laboratory, (1989), or Current Protocols in Molecular Biology, F. Ausubel et al., ed. Greene Publishing and Wiley-Interscience, New York (1987)). (See also PCT/US99/25200 for complexity management and other sample preparation techniques, which is hereby incorporated by reference in its entirety in their entireties for all purposes.) Where the single-stranded DNA population of the present invention is cDNA produced from a mRNA population, it may be produced according to methods known in the art. (See, e.g, Maniatis et al., supra, at 213-46.) In one embodiment, a sample population of single-stranded poly(A)+ RNA may be used to produce corresponding cDNA in the presence of reverse transcriptase, oligo-dT primer(s) and dNTPs. Reverse transcriptase may be any enzyme that is capable of synthesizing a corresponding cDNA from an RNA template in the presence of the appropriate primers and nucleoside triphosphates. In a preferred embodiment, the reverse transcriptase may be from avian myeloblastosis virus (AMV), Moloney murine leukemia virus (MMuLV) or Rous Sarcoma Virus (RSV), for example, and may be thermal stable enzyme (e.g., rTth DNA polymerase available from PE Applied Biosystems, Foster City, Calif.). In a preferred embodiment the RT is a SuperScript available from Invitrogen.

Multiple copies of RNA according to the present invention may be obtained by in vitro transcription from the DNA preferably using a polymerase such as, for example, T7 RNA polymerase in the presence of the appropriate nucleoside triphosphates.

In one embodiment of the present invention, the multiple copies of RNA may be labeled by the incorporation of biotinylated, fluorescently labeled or radiolabeled CTP or UTP during the RNA synthesis. See U.S. Pat. No. 5,800,992, 6,040,138 and International Patent Application PCT/US96/14839, which is expressly incorporated herein by reference. Alternatively, labeling of the multiple copies of RNA may occur following the RNA synthesis via the attachment of a detectable label in the presence of terminal transferase. In one embodiment, the detectable label may be radioactive, fluorometric, enzymatic, or colorimetric, or a substrate for detection (e.g., biotin). Other detection methods, involving characteristics such as scattering, IR, polarization, mass, and charge changes, may also be within the scope of the present invention.

In one embodiment, the amplified DNA or RNA of the present invention may be analyzed with a gene expression monitoring system. Several such systems are known. (See, e.g., U.S. Pat. No. 5,677,195; Wodicka et al., Nature Biotechnology 15:1359-1367 (1997); Lockhart et al., Nature Biotechnology 14:1675-1680 (1996), which are expressly incorporated herein by reference.) A preferred gene expression monitoring system according to the present invention may be a nucleic acid probe array, such as the GeneChip® nucleic acid probe array (Affymetrix, Santa Clara, Calif.). (See, U.S. Pat. Nos. 5,744,305, 5,445,934, 5,800,992, 6,040,193 and International Patent applications PCT/US95/07377, PCT/US96/14839, and PCT/US96/14839, which are expressly incorporated herein by reference. A nucleic acid probe array preferably comprises nucleic acids bound to a substrate in known locations. In other embodiments, the system may include a solid support or substrate, such as a membrane, filter, microscope slide, microwell, sample tube, bead, bead array, or the like. The solid support may be made of various materials, including paper, cellulose, gel, nylon, polystyrene, polycarbonate, plastics, glass, ceramic, stainless steel, or the like including any other support cited in 5,744,305 or 6,040,193. The solid support may preferably have a rigid or semi-rigid surface, and may preferably be spherical (e.g., bead) or substantially planar (e.g., flat surface) with appropriate wells, raised regions, etched trenches, or the like. The solid support may also include a gel or matrix in which nucleic acids may be embedded. The gene expression monitoring system, in one embodiment, may comprise a nucleic acid probe array (including an oligonucleotide array, a cDNA array, a spotted array, and the like), membrane blot (such as used in hybridization analysis such as Northern, Southern, dot, and the like), or microwells, sample tubes, beads or fibers (or any solid support comprising bound nucleic acids). See U.S. Pat. Nos. 5,770,722, 5,744,305, 5,677,195 5,445,934, and 6,040,193 which are incorporated here in their entirety by reference. (See also Examples, infra.) The gene expression monitoring system may also comprise nucleic acid probes in solution.

The gene expression monitoring system according to the present invention may be used to facilitate a comparative analysis of expression in different cells or tissues, different subpopulations of the same cells or tissues, different physiological states of the same cells or tissue, different developmental stages of the same cells or tissue, or different cell populations of the same tissue. (See U.S. Pat. Nos. 5,800,922 and 6,040,138.) In one embodiment, the proportional amplification methods of the present invention can provide reproducible results (i.e., within statistically significant margins of error or degrees of confidence) sufficient to facilitate the measurement of quantitative as well as qualitative differences in the tested samples. The proportional amplification methods of the present invention may also facilitate the identification of single nucleotide polymorphisms (SNPs) (i.e., point mutations that can serve, for example, as markers in the study of genetically inherited diseases) and other genotyping methods from limited sources. (See e.g., Collins et al., 282 Science 682 (1998), which is expressly incorporated herein by reference.) The mapping of SNPs can occur by any of various methods known in the art, one such method being described in U.S. Pat. No. 5,679,524, which is hereby incorporated by reference.

The RNA, single-stranded DNA, or double-stranded DNA population of the present invention may be obtained or derived from any tissue or cell source. Indeed, the nucleic acid sought to be amplified may be obtained from any biological or environmental source, including plant, virion, bacteria, fungi, or algae, from any sample, including body fluid or soil. In one embodiment blood is preferred. In one embodiment, eukaryotic tissue is preferred, and in another, mammalian tissue is preferred, and in yet another, human tissue is preferred. The tissue or cell source may include a tissue biopsy sample, a cell sorted population, cell culture, or a single cell. In a preferred embodiment, the tissue source may include brain, liver, heart, kidney, lung, spleen, retina, bone, lymph node, endocrine gland, reproductive organ, blood, nerve, vascular tissue, and olfactory epithelium. In yet another preferred embodiment, the tissue or cell source may be embryonic or tumorigenic.

Samples of nucleic acids may comprise unwanted nucleic acids that interfere with the analysis of target nucleic acids that are also present in the sample. The unwanted nucleic acids may be present in high levels and the target nucleic acids may be present in lower levels. Removal of the unwanted nucleic acids prior to amplification may be used to increase detection or improve analysis of target nucleic acids. In one embodiment the target nucleic acids are polyadenylated mRNA and target nucleic acids are amplified by first generating a first strand cDNA copy of the target mRNA using a primer that comprises a 3′ oligo dT region and a 5′ promoter sequence, such as T7, T3 or SP6. The first strand cDNA is converted to double stranded DNA with a functional double stranded promoter region. The promoter region may then be used to make multiple RNA copies of the starting mRNA using an RNA polymerase such as T7, T3 or SP6 polymerase.

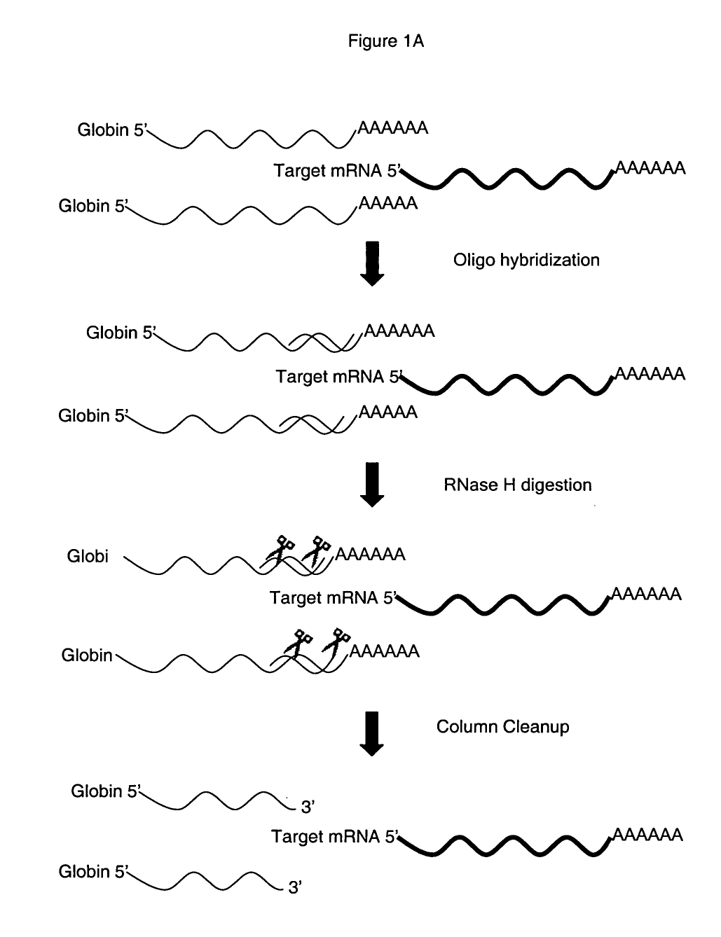

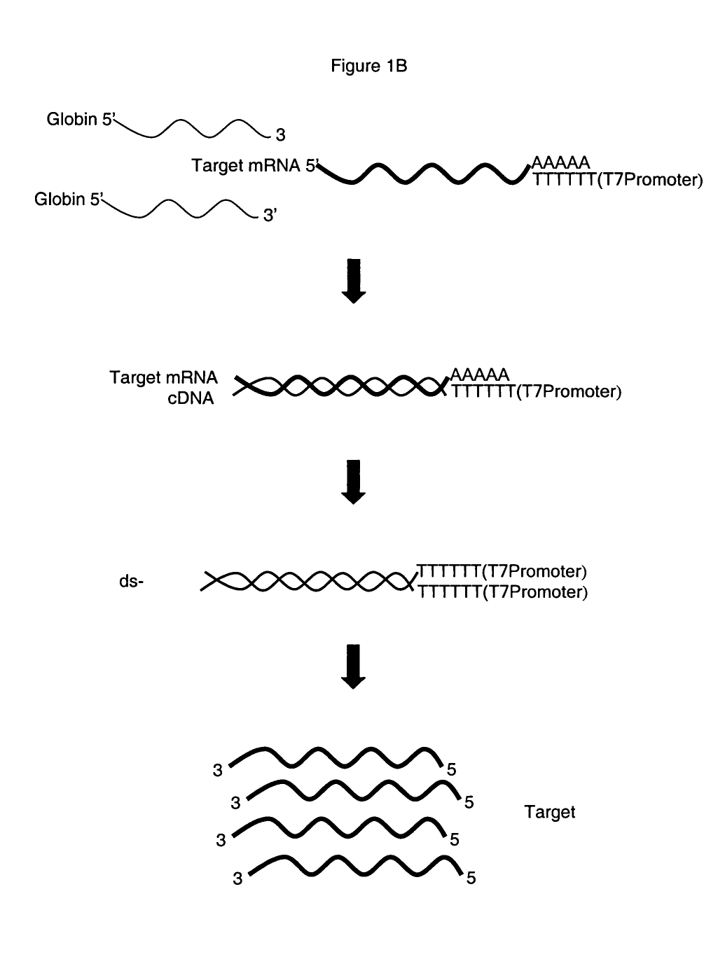

In one embodiment (see FIG. 1) a mixture of mRNA containing both target and non-target nucleic acid is mixed with a bait molecule, which may be a DNA oligonucleotide. The bait molecule is capable of hybridizing to the non-target nucleic acid but not to the target nucleic acid under standard hybridization conditions. In one embodiment the bait molecule is designed to hybridized within 20, 50, 100, 200, 500or 1000 bases of the 3′ end or poly(A) tail of the non-target nucleic acid. In one embodiment more than one bait molecule is used for each non-target molecule. In one embodiment the bait molecules hybridize near the 3′ end, upstream of the poly(A) tail.

The mixture is then treated with RNase H which digests RNA:DNA hybrids. Digestion of the RNA:DNA hybrid formed by the bait molecule and the non-target nucleic acid results in cleavage of the non-target nucleic acid. The RNase H may then be removed or inactivated. A primer comprising oligo dT and a promoter primer region is then added along with a reverse transcriptase. The primer hybridizes to the poly(A) tail and reverse transcriptase is used to generate a cDNA copy of the mRNA. Copies of the cleaved non-target nucleic acids will truncate at the location of cleavage so any sequence that is 5′ of the location of cleavage will not be copied, thus cleavage near the 3′ end effectively removes the non-target nucleic acid from the pool of mRNAs that will be efficiently amplified. The cDNA is then converted to double stranded DNA comprising a functional promoter region. An RNA polymerase is then used to generate multiple copies of the target nucleic acids.

In one embodiment the non-target nucleic acid is globin mRNA and the sample is from blood. Globin mRNA isolated from whole blood cells interferes with the hybridization process in microarray applications. Eliminating or reducing the generation of globin cRNAs prior to hybridization or analysis of mRNA isolated from whole blood cells reduces interference. Specifically, one or more DNA probes are designed to hybridize near the 3′-end (near the polyA site) of globin gene family mRNAs. Once the probe or probes is hybridized with the globin gene(s) RNAs, RNase H is used to degrade the RNAs within the RNA:DNA hybrid. As a result, during the generation of double-stranded cDNA by T7-poly(dT) primers, the globin gene mRNAs are not reversely transcribed efficiently; and thus little or no globin cRNA is produced. The globin genes code for proteins that transport oxygen through the bloodstream. The globin tetramer is the major constituent of the red blood cell. Globin genes are present as a gene family, different globin genes are expressed at different developmental stages. Typically in adults a tetramer is composed of two alpha-globin chains and two beta-globin chains along with an associated heme. There are also globin pseudogenes that may be targeted by GROs.

In a preferred embodiment the blood sample is isolated from a human and human globin genes are targeted. In other embodiments blood is isolated from another organism, for example, rat, mouse, dog, chicken, gorilla, or chimp. Reduction oligos are designed to be complementary to components of the hemoglobin family in the organism from which the blood is isolated. For example, if the blood sample being analyzed is from a dog the reduction oligos are designed to be complementary to dog genes, for example, dog globin genes. In one embodiment the methods are used to analyze blood samples after an organism has been treated or exposed to a particular drug, small molecule or stimuli. The toxicology of the treatment may be evaluated based on the resulting gene expression patern.

In one embodiment reduction oligos are designed that are complementary to a region in the 3′ region of one or more globin genes. The reduction oligos may be designed to have minimum homology to other mRNAs so that a reduction oligo does not hybridize efficiently to RNAs other than the unwanted RNAs.

In another embodiment the reduction oligo is modified at the 3′ end so that it is not capable of being extended by a polymerase. The reduction oligo may be blocked from extension at the 3′ end with, for example, a modified nucleotide. For a description of methods of blocking 3′ extension, see U.S. Ser. No. 09/854,317 the disclosure of which is incorporated herein by reference in its entirety.

In one embodiment the reduction oligonucleotide is modified to enhance the specificity or stability of its interaction with the unwanted RNA that it is targeted to. In another embodiment the conditions for hybridization are modified to optimize efficiency of the formation of the RNA:DNA complex between the probe and the unwanted nucleic acid. The reduction oligo may be double stranded in some portions.

In another embodiment conditions are modified to optimize RNase H cleavage of the RNA:DNA hybrid, for example, to efficiently eliminate globin gene(s) RNAs and to minimize non-specific degradation of other RNAs. For example, this step may be performed at a temperature range of between 37° C. and 70° C., more preferably at a temperature range of between 40° C. and 60° C. and more preferably at 50° C. A modified thermal stable form of RNase H may be used in one embodiment. Elevating the temperature of the cleavage reaction or using thermal cycling may be used to enhance cleavage. In one embodiment the reaction is cycled to allow for multiple annealing and cleavage reactions for a reduction oligo. A single reduction oligo may be used to cleave multiple RNAs. RNase H may be removed by any method known in the art, for example, the RNA may be purified after RNase H treatment, for example, by RNeasy purification, affinity purification, or phenol extraction.

In one embodiment mixtures of nucleic acids are depleted of RNAs from specific gene(s). The mixture may be any nucleic acid sample, for example, total RNA or poly (A) selected RNA.

In one embodiment unwanted RNAs are depleted or reduced from a sample containing total nucleic acid or total RNA and, following depletion, poly(A) RNA may be selected. Selection of poly(A) RNA following the nuclease treatment may be used to separate the RNA from the nuclease to eliminate the need for inactivation of the enzyme or a subsequent purification step, for example a column purification or organic extraction.

In one embodiment reduction oligonucleotides may be designed to hybridize to any region of an unwanted RNA and the complex of reduction oligonucleotide and unwanted RNA is removed. For example, the reduction oligonucleotide may be labeled with biotin and the complexes may be removed by incubation with streptavidin beads or on a streptavidin column. The reduction oligonucleotide may hybridize to any region of the unwanted RNA and multiple reduction oligonucleotides may be used for each unwanted RNA. The complexes may be removed by any method of affinity chromatography available. They may be bound to any form of solid support, resin, beads, glass, etc.

In one embodiment one or more mRNAs from a gene that is a member(s) of a gene family is depleted. RNAs from one or more members of a gene family may be depleted. A reduction oligo may be designed to hybridize to two or more members of a single gene family or the reduction oligo may be designed to hybridize to one member of a gene family and not to other members. Members of a gene family may share regions of homology so that a single oligonucleotide that is complementary to two or more members of a single gene family. Alternatively, an oligonucleotide may be designed to be complementary to one member of a gene family or one member of a group of related sequences and not complementary to other members of the gene family or of the group of related sequences.

In one embodiment mRNA that is a specific splice variant of a gene is depleted. For example a single gene may code for two or more different mRNAs. The mRNAs may differ as the result of alternative splicing. A probe may be designed to deplete one or more forms while leaving the other one or more forms intact.

In another embodiment specific variants of an mRNA are depleted. Multiple variant forms of mRNA may be generated from a single gene for a number of reasons, for example, alternative polyadenylation sites, alternative transcription start or stop sites, or post transcriptional modification. In addition an organism may have more than one copy of the same gene which vary at one or more locations. For example, diploid organisms typically carry two copies of each gene. The copies may be identical or they may differ at one or more polymorphic locations. DNA probes could be designed to hybridize to one polymorphic form and not to the other polymorphic form. For example, a gene for which an A and a B allele are present produces mRNAs that are specific for the A allele and mRNAs that are specific for the B allele. The A allele mRNAs could be selectively depleted from the sample by designing a reduction oligo that hybridizes to the A allele and not the B allele and digesting the A allele with RNase H.

In another embodiment the RNA that is depleted is not mRNA, for example rRNA, tRNA, snRNAs, snoRNAs, or RNaseP RNA may be depleted. See, for example, U.S. Pat. No. 6,613,516 which is incorporated herein by reference in its entirety.

The disclosed methods may be used to reduce representation of unwanted sequences in nucleic acid samples prior to amplification by any method known in the art. In one embodiment the method that is selected for amplification is a method that is generally unbiased. Some amplification methods are biased toward amplification of one region of the mRNA, for example, the 3′ end of the mRNA. In particular, the use of oligo(dT) to prime first strand synthesis may result in a bias toward the 3′ end of an mRNA, meaning that the 3′ end of some messages may be overrepresented relative to the 5′ end. A method that has reduced 3′ bias, for example, may employ random primers to synthesize cDNA. The cDNA may be end labeled or internally labeled and then hybridized to an array. For methods of unbiased amplification of nucleic acids see, for example, U.S. Pat. Nos. 6,495,320, 6,251,639 and 6,582,906 which are each incorporated herein by reference in their entireties.

When a method of amplification is used that does not employ priming mediated by a poly(A) tail at the 3′ end of the mRNA the method may employ multiple oligos for each unwanted mRNA. For example, if synthesis of the first strand cDNA is primer by random primers, the unwanted mRNAs may be targeted by multiple reduction oligos that hybridize throughout the unwanted mRNA. For example, several reduction oligos may be included for each unwanted mRNA. The reduction oligos may hybridize to different regions of the unwanted mRNA so that upon RNase H cleavage the unwanted mRNA is cleaved into three or more pieces. In one embodiment the reduction oligos may be extended by reverse transcriptase to generate longer regions of RNA:DNA hybrids that will result in longer regions of the unwanted RNA that will be digested.

The materials for use in the present invention are ideally suited for the preparation of a kit suitable for the amplification of nucleic acids. Such a kit may comprise reaction vessels, each with one or more of the various reagents, preferably in concentrated form, utilized in the methods. The reagents may comprise, but are not limited to the following: buffer, appropriate nucleotide triphosphates (e.g. dATP, dCTP, dGTP, dTTP; or rATP, rCTP, rGTP, and UTP) reverse transcriptase, RNase H, thermal stable DNA polymerase, RNA polymerase, and the appropriate primers and reduction oligos, for example reduction molecules specific for alphal, alpha 2 and beta globin. In addition, the reaction vessels in the kit may comprise 0.2-1.0 ml tubes capable of fitting a standard thermal cycler, which may be available singly, in strips of 8, 12, 24, 48, or 96 well plates depending on the quantity of reactions desired. Hence, the amplification of nucleic acids may be automated, e.g., performed in a PCR thermal cycler.

Also, the automated machine of the present invention may include an integrated reaction device and a robotic delivery system. In such cases, part of all of the operation steps may automatically be done in an automated cartridge. (See U.S. Pat. Nos. 5,856,174, 5,922,591, and 6,043,080.)

### EXAMPLES

Depletion of RNA

Total human RNA, 50 μg (50 μL of 1 μg/μL) is mixed on ice with 5 μL RNase-free H₂O, 10 μL 10×RNaseH buffer, 25 μL of a mixture of oligos at 1 μM each oligo (final concentration is 25 pmol each oligo), and 10 μL Hybridase™ thermostable RNase H (5 U/μL) (Epicenter, Madison Wis.) in a final volume of 100 μL. The mixture is incubated under the following conditions for 10 cycles: 70° C. for 2 min. then ramp 1° C. per sec. to 50° C., incubate at 50° C. for 5 min. then fast ramp to 70° C.

Following the incubation the RNase H is neutralized by adding 5 μL 0.5 M EDTA. The RNA is purified on an RNeasy mini column and eluted in 50 μL H₂O. Oligos are digested by adding 5.8 μL 10×DNase I buffer and 2 μL 10 U/μL DNase I and incubating at 37° C. for 20 min. DNase I is neutralize by adding 3 μL 0.5 M EDTA. The mixture is subjected to phenol/chloroform/isoamyl alcohol extraction with Phase-loc light.

The RNA is precipitated by adding 1 vol. 5M ammonium acetate, 1 μL pellet paint (or use glycogen) and 5 vol. absolute ethanol at room temp. The mixture is vortexed, microfuged at full speed for at least 5 min. then washed with 1×70% ethanol wash and air dry the pellet.

The procedure uses 0.5 pmol of each oligo and 1 U Hybridase per μg of total RNA. The RNase reaction may be scaled down, for example, 0.05 pmol each oligo per μg of total RNA may be used. 10×RNaseH buffer may be made with RNase-free ingredients (e.g. Ambion): 500 mM Tris, pH 7.5, 1 M NaCl and 100 mM MgCl₂. 10×DNaseI buffer is 400 mM Tris, pH 7.5 and 60 mM MgCl₂. DNaseI was from Amersham Pharmacia Biotech. Duplicate samples may be combined and the reactions scaled up, for example, during RNeasy cleanup (100 μg RNA capacity for RNeasy Mini) or during DNase digestion and subsequent extraction and precipitation.

### EXAMPLE 2

Depletion of Globin RNA from Blood.

Oligonucleotides were synthesized that were complementary to a region in the 3′ portion of each of the desired target mRNAs, for al: 5′-TGC AGG AAG GGG AGG AGG GGC TG-3′ (nt 512-534) (SEQ ID NO 1); for α2: 5′-TGC AAG GAG GGG AGG AGG GCC CG-3′ (nt 512-534) (SEQ ID NO 2) and for β5′-CCC CAG TTT AGT AGT TGG ACT TAG GG-3′ (nt 539-564) (SEQ ID NO 3). Oligos were HPLC-purified and were stored at −20° C. 10× Oligo Hyb Buffer was 100 mM Tris-HCl, pH 7.6 200 mM KCl and was stored at −20° C. 10×RNaseH Buffer, was 100 mM Tris-HCl, pH 7.6, 10 mM DTT and 20 mM MgCl₂ and was stored at −20° C. SUPERase.In™, 1 U/μL, 2500U an RNase inhibitor was purchased from Ambion (PN 2694). RNaseH, E.coli, 10 U/μL, 200U was also purchased from Ambion (PN 2292). EDTA at 0.5M was from Invitrogen (PN 750009) and the GeneChip® Sample Cleanup Module was from Affymetrix, Inc. (PN 900371).

Hybridization with Globin Reduction Oligos was done by preparing a 10× Globin Reduction Oligo Mix with the following final concentrations in the 10× mix: α1 Oligo at 7.5 AM, α2 Oligo at 7.5 μM and β Oligo at 20 μM. The hybridization mix was as follows: 3-10 μg total RNA from whole blood, 2 μl 10× Globin Reduction Oligo Mix, 1 μL 10× Oligo Hyb Buffer and nuclease free water to a final volume of 10 μL. The reaction was incubated in a thermal cycler at 70° C. for 5 minutes, and then cooled to 4° C.

RNaseH digestion immediately followed the cooling to 4° C. An appropriate amount of RNaseH (10 U/μL) was diluted 10-fold to 1 U/μL with 1×RNaseH Buffer. The RNaseH reaction mix was prepared as follows: 2 μL10×RNaseH Buffer, 1 AL SUPERase.In™ (1 U/μL), 2 μL diluted RNaseH (1 U/μL); 5 μL nuclease-free water for a total volume of 10 μL. 10 μL of the RNaseH reaction mix was added to each RNA:Globin Reduction Oligo hybridization sample and mixed thoroughly. The reaction was incubated at 37° C. for 10 minutes and cooled to 4° C. When the RNase reaction is complete, add 1 μL of 0.5M EDTA to each sample to stop the reaction and proceed immediately to the cleanup step. The RNaseH Digestion step was not left at 4° for an extended period of time because prolonged incubation may result in undesired nonspecific reduction of the sample.

RNaseH-Treated Total RNA Cleanup. The IVT cRNA Cleanup Spin Column from the GeneChip® Sample Cleanup Module (Affymetrix, Inc., Santa Clara, Calif.) were used to clean up the RNaseH-treated RNA samples. The recommended protocol for the Sample Cleanup Module was followed with the exception of the elution steps: 80 μL of RNase-free water was added to the processed sample prior to adding the cRNA Binding Buffer, 14 μL of RNase-free water was added to the center of the column, and spun at greater than or equal to 8,000×g (greater than or equal to 10,000 rpm) for 1 minute. The eluate was collected and applied again to the center of the column, and spun at greater than or equal to 8,000×g (greater than or equal to 10,000 rpm) for another minute. The final recovery volume after the second spin was approximately 13 IL and the recovery of the cleanup step is ˜75%. The eluate was collected and 10-11 μL of the treated total RNA sample was used in each reaction following the Standard GeneChip Target labeling Assay as described in the GeneChip Expression Analysis Technical Manual (available from Affymetrix, Inc., Santa Clara, Calif. and on the Affymetrix website). In one embodiment 1 μL of T7 Promoter Primer was used instead of the recommended 2 μL. Samples may also be stored at 4° C. or −20° C. for later use.

The IVT incubation time was extended from 4 hours to overnight (12-16 hours) in order to obtain sufficient material to hybridize onto the GeneChip arrays. With 5 μg of total RNA from PAXgene, more than 30 μg of labeled cRNA was routinely obtained following this protocol.

### Conclusion

The presently claimed invention provides a method for enriching nucleic acids. It is to be understood that the above description is intended to be illustrative and not restrictive. Many variations of the invention will be apparent to those of skill in the art upon reviewing the above description. The invention has been described primarily with reference to the enrichment and labeling of mRNA, but it will be readily recognized by those of skill in the art that the invention may be employed to enrich and label all types of nucleic acids including other forms of naturally and non-naturally occurring polynucleotides such as RNAs and DNAs. Furthermore, it will be understood by those of skill in the art that the enriched nucleic acids may be utilized in a wide variety of biological analyses in no way limited to those methods disclosed in the present invention. Therefore, it is to be understood that the scope of the invention is not to be limited except as otherwise set forth in the claims.

1. A method for amplifying a nucleic acid sample from blood comprising:
providing a nucleic acid sample from blood;
hybridizing at least one reduction oligonucleotide to at least one unwanted RNA in the sample;
incubating the mixture with an RNase H and subsequently inactivating the RNase H;
hybridizing a primer comprising oligo dT to the RNA in the mixture;
extending the primer to make cDNA; and
amplifying the cDNA.

2. The method of claim 1 wherein the unwanted RNA comprises a poly(A) tail and wherein the reduction oligonucleotide hybridizes to the unwanted RNA in the region of the unwanted RNA that is near the 5′ end of the poly(A) tail of the unwanted RNA.

3. The method of claim 1 wherein the RNase H is inactivated by depleting RNase H from the mixture.

4. The method of claim 1 wherein the RNase H is thermolabile and inactivation is by heating

5. The method of claim 1 wherein the RNase H is inactivated by addition of EDTA to the mixture.

6. The method of claim 1 wherein the RNase H is inactivated by separating the RNase H from the nucleic acid by organic extraction.

7. The method of claim 1 wherein the RNase H is removed by separating the RNA from the RNase H by column purification.

8. The method of claim 1 wherein the primer further comprises a RNA polymerase promoter sequence.

9. The method of claim 8 wherein the step of amplifying the cDNA comprises making double stranded cDNA comprising a functional RNA polymerase promoter region and synthesizing multiple copies of RNA from the double stranded cDNA using an RNA polymerase.

10. The method of claim 1 wherein the unwanted nucleic acid is a globin mRNA.

11. The method of claim 1 wherein the unwanted nucleic acid is selected from the group consisting of alpha-1 globin, alpha-2 globin and beta globin.

12. The method of claim 10 wherein a plurality of different species of reduction oligonucleotides are used and each species is complementary to a globin mRNA.

13. The method of claim 1 wherein after hybridizing the reduction oligonucleotide to the unwanted mRNA, the reduction oligonucleotide is extended by a polymerase.

14. The method of claim 1 wherein after incubating the mixture with RNase H the reduction oligonucleotide is removed.

15. The method of claim 1 wherein the at least one reduction oligonucleotide consists essentially of SEQ ID NO 1.

16. The method of claim 1 wherein the at least one reduction oligonucleotide consists essentially of SEQ ID NO 2.

17. The method of claim 1 wherein the at least one reduction oligonucleotide consists essentially of SEQ ID NO 3.

18. The method of claim 1 wherein a mixture of different sequence reduction oligonucleotides are added to the mixture.

19. The method of claim 18 wherein the mixture comprises SEQ ID NOs 1, 2and3.

20. The method of claim 1 wherein said nucleic acid sample from blood is obtained from blood that was collected in a container containing an RNA stabilizing agent.

21. The method of claim 20 wherein said RNA stabilizing agent is selected from the group consisting of cationic compounds, detergents, chaotropic salts, ribonuclease inhibitors, chelating agents, and mixtures thereof.

22. The method of claim 20 wherein said RNA stabilizing agent is selected from the group consisting of phenol, chloroform, acetone, alcohols and mixtures thereof.

23. The method of claim 20 wherein said nucleic acid sample from blood is obtained from blood that was collected in a container containing a RNA stabilizing agent and wherein said RNA stabilizing agent is selected from the group consisting of mercapto-alcohols, di-thio-threitol (DTT) and mixtures thereof.

24. A method for analyzing a nucleic acid sample isolated from blood comprising:
a. providing a first nucleic acid sample obtained from a blood sample;
b. blocking amplification of globin mRNA sequences in the nucleic acid sample by hybridizing a reduction oligonucleotide to said globin mRNA sequences to form a RNA:DNA hybrid and digesting the RNA:DNA hybrid;
c. amplifying unblocked nucleic acid sequences to produce an amplified nucleic acid sample;
d contacting said amplified nucleic acid sample with a solid support comprising nucleic acid probes to generate a hybridization pattern; and
e. analyzing the hybridization pattern.

25. The method of claim 24, further comprising: detecting the presence or absence of hybridization of said amplified nucleic acid sample to said nucleic acid probes on said solid support.

26. The method of claim 24, further comprising: labeling said amplified nucleic acid sample.

27. (canceled)

28. The method of claim 24 wherein said unblocked nucleic acid sequences are non-specifically amplified by in vitro transcription.

29. (canceled)

30. The method of claim 24 wherein said globin mRNAs are greater than 20% of the first nucleic acid sample and wherein said globin mRNAs are less than 20% of the amplified nucleic acid sample.

31. A method for amplifying a nucleic acid sample from blood comprising:
providing a nucleic acid sample from blood;
hybridizing at least one reduction oligonucleotide to at least one globin mRNA in the sample generating reduction oligonucleotide: globin mRNA complexes;
removing said complexes from the sample; and,
amplifying at least one target RNA remaining in the sample.

32. The method of claim 31 wherein said complexes are removed from the sample by affinity purification.

33. The method of claim 31 wherein said reduction oligonucleotide comprises biotin and said complexes are removed from the sample by hybridization to a solid support.

34. The method of claim 33 wherein said solid support comprises streptavidin.

35. The method of claim 31 wherein the RNA is amplified by mixing with random primers, extending the random primers to make cDNA and labeling the cDNA.

36. The method of claim 35 wherein the labeled cDNA is hybridized to a solid support and the hybridization pattern is analyzed.

37. A method of analyzing a nucleic acid sample from a blood sample comprising:
amplifying mRNA from the nucleic acid sample to generate an amplified sample wherein amplification of globin mRNA is blocked during said amplifying step;
labeling said amplified sample;
hybridizing the amplified sample to an array of nucleic acid probes to generate a hybridization pattern; and
analyzing the hybridization pattern.

38. The method of claim 37 wherein said amplifying step comprises hybridizing an extendable primer comprising oligo dT to said nucleic acid sample, extending said primer with a reverse transcriptase to make cDNA and amplifying said cDNA.

39. The method of claim 38 wherein amplification of globin mRNA is blocked by hybridization of one or more blocking molecules to one or more globin mRNA transcripts prior to extending said extendable primer with reverse transcriptase, wherein said one or more blocking molecules hybridize to said one or more globin mRNA transcripts and block reverse transcription of said globin mRNA transcripts.

40. The method of claim 38 wherein said one or more blocking molecules are peptide nucleic acids.

41. The method of claim 39 wherein said one or more blocking molecules hybridize to a globin mRNA selected from the group consisting of alpha-1 globin, alpha-2 globin and beta globin.

42. The method of claim 37 wherein the hybridization pattern is analyzed to determine an expression profile for said nucleic acid sample.

43. The method of claim 37 wherein said nucleic acid sample is isolated from a blood sample that was collected in a container containing an RNA stabilizing agent selected from the group consisting of cationic compounds, detergents, chaotropic salts, ribonuclease inhibitors, chelating agents, phenol, chloroform, acetone, alcohols, mercapto-alcohols, di-thio-threitol (DTT), and mixtures thereof.
